(19) United States
(12) Patent Application Publication
(10) Pub. No.: US 2014/0277449 A1
(43) Pub. Date: Sep. 18, 2014

(54) BONE SCREW AND TENDON FIXATION ENHANCEMENT DEVICES AND METHODS

(71) Applicant: Woven Orthopedic Technologies, LLC., Manchester, CT (US)

(72) Inventor: A. ALEXANDER JONES, Savannah, GA (US)

(73) Assignee: Woven Orthopedic Technologies, LLC., Manchester, CT (US)

(21) Appl. No.: 14/209,514

(22) Filed: Mar. 13, 2014

Related U.S. Application Data
(60) Provisional application No. 61/801,880, filed on Mar. 15, 2013.
(60) Provisional application No. 61/802,004, filed on Mar. 15, 2013.
(60) Provisional application No. 61/802,083, filed on Mar. 15, 2013.

(51) Int. Cl. A61F 2/08

(52) U.S. Cl. CPC A61F 2/0811
USPC 623/13.14; 606/300

(57) ABSTRACT

A sleeve system to support surgical screws or prosthetic devices on, against, or within a bone or tissue of a subject includes a woven sleeve having a first end, a second end, and an elongated body. The elongated body defines an interior portion of the woven sleeve. The first end has an aperture allowing access to the interior portion. The woven sleeve is formed from a plurality of suture material, and the woven sleeve may be coated, impregnated, or at least partially filled with a material that facilitates integration of the woven sleeve with the bone or tissue.

### CROSS-REFERENCE TO RELATED APPLICATIONS

This application claims priority to U.S. Provisional Application Nos. 61/801,880, filed Mar. 15, 2013; 61/802,004, filed Mar. 15, 2013; and 61/802,083, filed Mar. 15, 2013, the disclosures of which are incorporated herein by reference in their entirety.

### TECHNICAL FIELD OF THE INVENTION

This invention generally relates to devices and methods for engagement, fixation, and/or attachment of orthopedic fasteners or tendons to bone.

### BACKGROUND OF THE INVENTION

U.S. Pat. No. 5,984,926, issued on Nov. 16, 1999, which is incorporated herein in its entirety by reference, describe various problems associated with achieving adequate grip or achieving adequate bone screw purchase strength in cancellous bone or osteopenic/osteoporotic bone or bone where the fractures or poor quality bone preclude optimal engagement of bone. U.S. Pat. No. 5,984,926 also discloses a suture based device for saving screw holes in bone. U.S. Patent Application Publication No. 2012/0239145, which is incorporated herein in its entirety by reference, discloses a suture based method for connecting together sections of tendon.

Because of the variety of types of injuries, bone conditions and anatomical variations and limitations, there remains a need for devices that improve screw purchase strength in a variety of clinical situations. Known methods, such as the method described in U.S. Patent Application Publication No. 2007/0118144 A1 to Truckai et al., incorporated herein in its entirety by reference, deliver fluidized materials through a trocar suffer the limitation that the materials being delivered cannot be precisely contained within the bone. Devices and methods discussed in U.S. Pat. No. 8,366,711 to Rabiner et al., incorporated herein in its entirety by reference, while using a trocar or trocar-type device for delivery are too limited in that the reinforcing material is bounded by a balloon.

### BRIEF SUMMARY OF THE INVENTION

According to an embodiment of the current invention, a sleeve system to support surgical screws or prosthetic devices on, against, or within a bone or tissue of a subject is provided. The sleeve system may include a woven sleeve having a first end, a second end, and an elongated body defining an interior portion of the woven sleeve. The first end may have an aperture allowing access to the interior portion, and the woven sleeve may be formed from a plurality of suture material. The woven sleeve may be coated, impregnated, or at least partially filled with a material that facilitates integration of the woven sleeve with the bone or tissue.

According to another embodiment of the current invention, a method of creating a support system for surgical screws or prosthetic devices on, against, or within a bone or tissue of a subject is provided. The method includes providing a woven sleeve having a first end, a second end, and an elongated body defining an interior portion. The first end may include an aperture allowing access to the interior portion of the woven sleeve, and the woven sleeve can be formed from a plurality of suture material. The method may also include providing a trocar with an aperture through which the woven sleeve can be inserted, and delivering the woven sleeve through the trocar to a desired location on, against, or within a bone or tissue, or between two sections of bone or tissue. The method may also include allowing a cement or an adhesive on the woven sleeve to solidify.

According to another embodiment of the current invention, a method of attaching a tendon to a bone of a subject is provided. The method may include providing a woven sleeve having a first end, a second end, and an elongated body defining an interior portion. The first end may include an aperture allowing access to the interior portion of the woven sleeve, and the woven sleeve can be formed from a plurality of suture material. The method may also include delivering at least the first end of the woven sleeve to a desired location on, against, or within a bone or tissue, or between two sections of bone or tissue, and filling at least a portion of the woven sleeve with a material to secure the first end of the woven sleeve in the desired location. The method further includes attaching the tendon of the subject to a portion of the woven sleeve that extends outward from the desired location.

Additional features, advantages, and embodiments of the invention are set forth or apparent from consideration of the following detailed description, drawings and claims. Moreover, it is to be understood that both the foregoing summary of the invention and the following detailed description are exemplary and intended to provide further explanation without limiting the scope of the invention as claimed.

### BRIEF DESCRIPTION OF DRAWINGS

The accompanying drawings, which are included to provide a further understanding of the invention and are incorporated in and constitute a part of this specification, illustrate preferred embodiments of the invention and together with the detailed description serve to explain the principles of the invention. In the drawings:

FIG. 1A is a perspective view of a sleeve according to one embodiment of the present invention.

FIG. 1B shows a cross-section of a first end of the sleeve shown in FIG. 1A.

FIG. 2 illustrates sheets made with a composite material according to one embodiment of the present invention.

FIG. 3 is a perspective view illustrating the delivery of a filled woven sleeve through a trocar to a desired location according to one embodiment of the present invention.

FIG. 4 illustrates an extension of suture material from a hole for the purpose of attaching the suture material to a tendon, as well as a screw that may be used to fasten the suture material when filled, or unfilled as described above, according to embodiments of the present invention.

### DETAILED DESCRIPTION OF THE INVENTION

Some embodiments of the current invention are discussed in detail below. In describing embodiments, specific terminology is employed for the sake of clarity. However, the invention is not intended to be limited to the specific terminology and examples selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated. All references cited in this specification are incorporated herein by reference.

FIG. 1A shows a woven sleeve 10 with an elongated body 12 having an interior portion 14. The body 12 includes a first end 16 and a second end 18. The first end 16 includes an aperture 20 (FIG. 1B) that provides access to the interior portion 14 of the woven sleeve. The second end 18 of the woven sleeve 10 may be closed or partially closed. For example, the sleeve 10 may be tapered such that an aperture 22 of the second end 18 is closed or narrowed relative to the aperture 20 of the first end 16. Additionally, the sleeve 10 may be impregnated with a suitable adhesive or bone cement 26 to add thickness to the sleeve and rigidity to the assembly when cured. For example, the adhesive or bone cement 26 can be impregnated into the woven structure of the sleeve 10 before implanting into the bone or tissue of a subject, and can remain uncured until placed in the desired position. Alternatively, the bone cement or adhesive 26 can be added to or impregnated into the sleeve 10 in situ. In some embodiments, the bone cement or adhesive 26 is added after the sleeve is implanted into the subject.

FIG. 2 shows a sheet 30 of fabric used to create the body 12 of the woven sleeve 10 in FIGS. 1A and 1B. The sheet 30 may be woven from a plurality of components 24, such as, for example, suture material. However, the sleeve 10 may also be woven from other threads, fibers, or materials suitable for use in the body of a patient. As shown in FIG. 1B, the woven sleeve may be filled with one or more of various materials 28, including a morselized bone graft, a bone growth-stimulating agent, such as a bone morphogenic protein (BMP), and/or an antibiotic agent to provide protection against infection. The woven sleeve may also, or alternatively, be filled with uncured an adhesive or bone cement, which can be in liquid or gel form. In some embodiments, the sheet 30 may be used as a sheet (i.e., without being rolled into a sleeve) for filling irregular shapes or reinforcing connections such as tendon reattachment. While the sheet 30 may be rolled into a sleeve, such as that shown in FIG. 1, the sleeve 10 can also be formed by weaving or interconnecting the plurality of components 24 into a pre-formed sleeve shape.

As shown in FIG. 3, the sleeve 10 may then be delivered to a desired location, such as an area within or surrounding an open fracture in a bone 32 or within a bone 32, through an aperture 34 created in the bone 32 for accepting a trocar 36. The filled woven sleeve 10 is then delivered through the trocar 36 to a desired location on, against, or within a bone or between two sections of bone where it is allowed to solidify and integrate with surrounding bone and tissue.

A screw (see screw 40 in FIG. 4) or other surgical securing means may be inserted within the space 14 of the sleeve 10 of the above embodiments, and thereby placed and firmly held within a bone or tissue of the subject.

FIG. 4 shows a woven sleeve 100 according to another embodiment of the invention. The woven sleeve 100 can include the various aspects of the woven sleeve described in the above embodiments, and such aspects will not be repeated here. In FIG. 4, the woven sleeve 100 extends outward from the aperture 34 formed in the bone 32 so that the sleeve 100 can be attached to a tendon (not pictured), or other tissue or device of a patient. The sleeve may form a loop 38 exterior to the bone 32, as shown by the sleeve 100 in FIG. 4. The loop 38 may facilitate attachment of the sleeve 100 to a tendon, for example.

After the sleeve 100 is delivered to the desired location, such as an area within or surrounding an open fracture in a bone or within a bone, through the aperture created in the bone, the sleeve 100 may be filled with a fill material 28 that is used to anchor the suture sleeve 100 to the bone 32. As discussed above, the fill material 28 may be morselized bone graft, a bone growth-stimulating agent, such as a bone morphogenic protein (BMP), and/or an antibiotic agent to provide protection against infection, and/or an adhesive, the adhesive preferably being in liquid or gel form. The sleeve 100 may become enlarged below the surface of the bone 32 due to the addition of the fill material 28, so as to prevent the sleeve 100 from pulling out of the hole 34 once the sleeve 100 is filled. Then, the sleeve 100 extending from the bone 32 may be attached to the tendon, or other structure, thus reattaching the tendon to the bone 32. In addition, or alternatively, a screw 40 may be inserted through the sleeve 100 to the desired location within the bone or tissue. The length of the sleeve is selected such that sufficient suture material will extend from the hole so as to allow attachment of the tendon to the suture.

The sleeve 100 does not necessarily need to be formed in a loop 38, and can instead be a single segment of sleeve 100′, according to an alternative embodiment shown in FIG. 4.

The above-described methods can serve to secure a tendon to a bone. The tendon re-attachment is done by providing a woven sleeve with an elongated body having an interior portion, the body having a first end and a second end, the first end having an aperture allowing access to the interior portion of the woven sleeve, woven sleeve being formed from a plurality of suture material and the second end of the woven sleeve being closed or at least partially closed. The sleeve is inserted into the hole and filled.

According to the above descriptions and related drawings, embodiments of the present invention can include, for example, the following features.

In one embodiment, a sleeve system to support surgical screws or prosthetic devices on, against, or within a bone or tissue of a subject is provided. The sleeve system includes a woven sleeve 10 having a first end 16, a second end 18, and an elongated body 12 defining an interior portion 14 of the woven sleeve 10. The first end 16 has an aperture 20 allowing access to the interior portion 14. The second end 18 of the woven sleeve 10 can be at least partially closed or tapered. The woven sleeve 10 can be formed from a plurality of components 24, such as suture material, that is woven or interconnected. The woven sleeve 10 may be coated, impregnated, or at least partially filled with one or more materials 26, 28 that facilitate integration of the woven sleeve 10 with the bone 32 or tissue of a subject. The materials 26, 28 can include at least one of an adhesive, a bone cement, a bone growth-stimulating agent, an antibiotic agent, and a morselized bone graft. One or more of these materials 26, 28 can fill at least a portion of the space 14 of the sleeve 10, or can coat or infiltrate the woven or interconnected components 24 of the sleeve 10. In some embodiments, one or more of the materials 26, 28 may only be provided in a portion of the sleeve 10 that is within a bone 32 or other tissue of the subject.

The sleeve system may also include a trocar 36 having an aperture through which the woven sleeve 10 is deliverable to a desired location on, against, or within the bone 32 or tissue, or between two sections of bone or tissue. The sleeve 10 may be delivered through the trocar 36 with one or more of the materials 26, 28 applied to the sleeve. Additionally, one or more of the materials 26, 28 inserted into the sleeve 10, via the trocar 36, after the sleeve 10 is in place in the desired position.

According to an embodiment, the woven sleeve may be attachable to a tendon of the subject. For example, a portion of the sleeve 100, 100′ may extend outward from the desired location on, against, or within the bone or tissue, or between the two section of bone or tissue, to attach to the tendon. At least a portion of the woven sleeve 100 that extends outward from the desired location can be formed as a loop 38. Alternatively, the sleeve 100′ may be formed as a single segment without a loop.

According to an embodiment of the present invention, a method of creating a support system for surgical screws or prosthetic devices on, against, or within a bone or tissue of a subject is provided. The method includes providing a woven sleeve 10 in accordance with the above-described embodiments of the woven sleeve. The method also includes providing a trocar 36 with an aperture through which the woven sleeve 10 can be inserted, and delivering the woven sleeve 10 through the trocar 36 to a desired location on, against, or within a bone 32 or tissue, or between two sections of bone or tissue. Additionally, the method may include allowing a cement or an adhesive on the woven sleeve to solidify.

According to an embodiment, the method may also include applying the cement or the adhesive to the woven sleeve 10 prior to delivering the woven sleeve 10 through the trocar 36.

According to another embodiment of the present invention, a method of attaching a tendon to a bone of a subject is provided. The method includes providing a woven sleeve 100 or 100′ having a first end 16, a second end 18, and an elongated body 12 defining an interior portion 14. The first end 16 has an aperture 20 that allows access to the interior portion 14 of the woven sleeve 100, 100′. The woven sleeve 100, 100′ may be formed from a plurality of components 24, such as suture material, that are woven or interconnected to each other. In some embodiments, the sleeve 100, 100′ may be formed by rolling a sheet 30 of such woven or interconnected components 24. The method also includes delivering at least the first end 16 of the woven sleeve 100, 100′ to a desired location on, against, or within a bone 32 or tissue, or between two sections of bone or tissue, and filling at least a portion of the woven sleeve 100, 100′ with a material 28 to secure the second end 18 of the woven sleeve 100, 100′ in the desired location. Additionally, the method includes attaching the tendon of the subject to a portion of the woven sleeve 100, 100′ that extends outward from the desired location. The portion of the woven sleeve 100, 100′ that extends outward can include the first end 16. According to an embodiment, the portion of the woven sleeve 100 that extends outward from the desired location can be formed as a loop 38. The loop 38 may be a pre-formed loop of the woven sleeve 100. Alternatively, the loop 38 may be formed by affixing one portion of the woven sleeve 100 to another portion of the woven sleeve. In another embodiment, the woven sleeve may include one or more fibers or suture material that forms a “tail” of the woven sleeve, and the tail can be a pre-formed loop or tied to form a loop. In yet another embodiment, the portion of the woven sleeve 100′ that extends outward from the desired location can be a single segment.

The attaching the tendon of the subject to a portion of the woven sleeve 100, 100′ that extends outward from the desired location may be achieved by directly attaching the woven sleeve 100, 100′ to the tendon. For example, the tendon can be attached to the loop 38 of woven sleeve 100 or the single segment of woven sleeve 100′. The attaching of the tendon to the sleeve can be performed via a suture, for example. According some embodiments, the tendon may be attached to either an interior or an exterior of the sleeve. In another embodiment, the tendon may be attached by positioning the tendon into the interior portion of the sleeve such that the sleeve grips the tendon. For example, a pulling of the tendon on the woven sleeve may cause the woven sleeve to tighten around the tendon due to the axial tension in the woven sleeve.

According to some embodiments, at least one of a morselized bone graft, a bone growth-stimulating agent, such as a bone morphogenic protein (BMP), an antibiotic agent, and/or an adhesive may be placed within the bone, tissue, or space between bone and/or tissue prior to inserting the sleeve into the desired location. In this way, securing of the sleeve and healing of the bone or tissue, for example, may be aided.

According to one embodiment of the present invention, it has been discovered that the problems left unanswered by known art can be solved by providing a method for creating support for surgical screws and prosthetic device by providing a woven sleeve with an elongated body having an interior portion, the body having a first end and a second end, the first end having an aperture allowing access to the interior portion of the woven sleeve, woven sleeve being formed from suture material; saturating the woven sleeve with bone cement or adhesive, the cement or adhesive preferably being fluid form such as in liquid or gel form; and allowing the filled woven sleeve and its contents to solidify to form a matrix from the cement or adhesive that is reinforced by the suture material. Solidification may be through the use of catalysts or UV or other light sources, depending on the type of adhesive used, when adhesives are used as part of the material filling the woven sleeve.

According to another embodiment of the present invention, a method is provided for creating support for surgical screws and prosthetic device by providing a woven sleeve with an elongated body having an interior portion, the body having a first end and a second end, the first end having an aperture allowing access to the interior portion of the woven sleeve, woven sleeve being formed from a plurality of suture material and the second end of the woven sleeve being closed or at least partially; filling the woven sleeve with morselized bone graft, a bone growth-stimulating agent, such as a bone morphogenic protein (BMP), and/or an antibiotic agent to provide protection against infection, and/or an adhesive, the adhesive preferably being in liquid or gel form; providing a trocar that can accept the filled woven sleeve to a desired location on, against, or within a bone or between two sections of bone; delivering the filled woven sleeve through the trocar to a desired location on, against, or within a bone or between two sections of bone; and allowing the filled woven sleeve and its contents to solidify. Solidification may be through the use of catalysts or UV or other light sources, depending on the type of adhesive used, when adhesives are used as part of the material filling the woven sleeve.

According to another embodiment of the present invention, a method is provided for creating support for surgical screws and prosthetic device by providing a woven sleeve with an elongated body having an interior portion, the body having a first end and a second end, the first end having an aperture allowing access to the interior portion of the woven sleeve, woven sleeve being formed from a plurality of suture material and the second end of the woven sleeve being closed or at least partially closed; and attaching the woven sleeve to a section of bone through the use of a screw and thus attaching the sleeve directly to the screw or to a looped retainer that is attached to the screw or screws. Alternatively, the woven sleeve may be first fixed to the bone by making a hole in the bone, inserting the closed end of the sleeve into the hole, and filling the woven sleeve with morselized bone graft, a bone growth-stimulating agent, such as a bone morphogenic protein (BMP), and/or an antibiotic agent to provide protection against infection, and/or an adhesive, the adhesive preferably being in liquid or gel form, so that the sleeve becomes enlarged below the surface of the bone with the fill material, so as to prevent the sleeve from pulling out of the hole once filled, and then attaching the suture extending from the bone to the tendon, and thus reattaching the tendon to the bone. The filling of the sleeve may be accomplished by providing a trocar that can accept the filled woven sleeve to a desired location on, against, or within a bone or between two sections of bone; delivering the filled woven sleeve through the trocar to a desired location on, against, or within a bone or between two sections of bone; and then allowing the filled woven sleeve and its contents to solidify. Solidification may be through the use of catalysts or UV or other light sources, depending on the type of adhesive used, when adhesives are used as part of the material filling the woven sleeve.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

I hereby claim:

1. A sleeve system to support surgical screws or prosthetic devices on, against, or within a bone or tissue of a subject, comprising:
a woven sleeve having a first end, a second end, and an elongated body defining an interior portion of the woven sleeve, the first end having an aperture allowing access to the interior portion, the woven sleeve being formed from a plurality of suture material,
wherein the woven sleeve is configured to be coated, impregnated, or at least partially filled with a material that facilitates integration of the woven sleeve with the bone or tissue.

2. The sleeve system of claim 1, wherein the material is at least one of an adhesive, a bone cement, a bone growth-stimulating agent, an antibiotic agent, and a morselized bone graft.

3. The sleeve system of claim 1, further comprising a trocar having an aperture through which the woven sleeve is deliverable to a desired location on, against, or within the bone or tissue, or between two sections of bone or tissue.

4. The sleeve system of claim 1, the second end of the woven sleeve being at least partially closed.

5. The sleeve system of claim 1, wherein the woven sleeve is configured to attach to a tendon of the subject, and is configured to extend outward from the desired location on, against, or within the bone or tissue, or between the two section of bone or tissue, to attach to the tendon.

6. The sleeve system of claim 1, wherein at least a portion of the woven sleeve that extends outward from the desired location is formed as a loop.

7. A method of creating a support system for surgical screws or prosthetic devices on, against, or within a bone or tissue of a subject, comprising:
providing a woven sleeve having a first end, a second end, and an elongated body defining an interior portion, the first end having an aperture allowing access to the interior portion of the woven sleeve, the woven sleeve being formed from a plurality of suture material;
providing a trocar with an aperture through which the woven sleeve can be inserted;
delivering the woven sleeve through the trocar to a desired location on, against, or within a bone or tissue, or between two sections of bone or tissue; and
allowing a cement or an adhesive on the woven sleeve to solidify.

8. The method according to claim 7, further comprising applying the cement or the adhesive to the woven sleeve prior to delivering the woven sleeve through the trocar.

9. A method of attaching a tendon to a bone of a subject, comprising:
providing a woven sleeve having a first end, a second end, and an elongated body defining an interior portion, the first end having an aperture allowing access to the interior portion of the woven sleeve, the woven sleeve being formed from a plurality of suture material;
delivering at least the first end of the woven sleeve to a desired location on, against, or within a bone or tissue, or between two sections of bone or tissue;
filling at least a portion of the woven sleeve with a material to secure the first end of the woven sleeve in the desired location; and
attaching the tendon of the subject to a portion of the woven sleeve that extends outward from the desired location.

10. The method of claim 9, wherein the portion of the woven sleeve that extends outward from the desired location is formed as a loop.
